(19) United States
(12) Patent Application Publication
(10) Pub. No.: US 2025/0187005 A1
(43) Pub. Date: Jun. 12, 2025

(54) MICROFLUIDIC SYSTEM AND METHOD

(71) Applicant: The Board of Trustees of the University of Illinois, Urbana, IL (US)

(72) Inventors: Hua Gao, Chicago, IL (US); Gopakumar Kamalakshakurup, Windsor, WI (US); Ian Papautsky, Willowbrook, IL (US); Jian Zhou, Chicago, IL (US)

(21) Appl. No.: 18/845,876

(22) PCT Filed: Mar. 1, 2023
(86) PCT No.: PCT/US23/14229

§ 371 Date: Sep. 10, 2024

Related U.S. Application Data
(60) Provisional application No. 63/320,144, filed on Mar. 15, 2022.

(51) Int. Cl. B01L 3/00

(52) U.S. Cl. CPC B01L 3/502761; B01L 3/50273; B01L 2200/0647; B01L 2300/0645; B01L 2300/0654; B01L 2300/088; B01L 2400/0487

(57) ABSTRACT

A microchannel includes a single inlet and a single outlet, a spiral section downstream from the single inlet, a straight section downstream from the spiral section, a detection section downstream from the straight section, and an expansion section downstream from the detection section and disposed between the detection section and the single outlet. The microchannel is to receive fluid having particles. In addition, at least the straight section and the detection section are configured to orient particles within the detection section in an area away from sidewalls of the detection section and into one of a single particle stream or two particle streams. The two particle streams immediately adjacent to each other appear as a single particle stream for optimized focusing and orientation of the particles in a focused stream within the microchannel.

### CROSS-REFERENCE TO PRIOR APPLICATION

This International Application claims the benefit of U.S. Provisional Application Ser. No. 63/320,144 filed on Mar. 15, 2022. The entire contents of this application is incorporated herein by reference in its entirety.

### FIELD OF DISCLOSURE

The present disclosure relates generally to microfluidic chips and, more specifically, to a microfluidic system and method for optimized focusing and orientation of particles within a microchannel of the microfluidic system.

### BACKGROUND

A wide range of devices has been introduced for microfluidic sorting of cells and/or microparticles. Specifically, it is often desired to separate various particles or cells from the sample fluid mixture, such as the separation of viable and motile sperm from non-viable and non-motile sperm or the separation of sperm by gender. Precise manipulation of particle position inside microscale flow enables highly efficient sorting of particles, if differential markers exist. Specifically, spatial differentiation of particles or cells can be achieved by taking advantage of hydrodynamic forces due to the physical structure of the microfluidic channel or the intense interaction between particles suspended in flow.

One of the most prominent technics is inertial microfluidics. Inertial microfluidics is a label-free approach that leverages hydrodynamic forces acting on cells suspended in flow and the inertia of the carrier fluid to sort cells based on their physical phenotype (primarily size, but also shape and deformability). In this approach, cells migrate to focusing positions under the influence of these hydrodynamic forces. Spiral is the most frequently used channel geometry for inertial focusing microfluidic chips. Focusing quality and 3D confinement of cells is the major concern in these devices. For example, a wider stream width (i.e., poor focusing quality) can be reduced with an increased flow rate of fluid. Focusing quality and efficiency can also be impacted by sharp turns and abrupt changes in depth or width of channels, and can lead not only to degraded performance, but also chip clogging. Most commonly, these spiral inertial microfluidic chips are fabricated in polydimethylsiloxane (PDMS) due to simplicity and low cost. However, the microfluidic chips comprising of the PDMS material are incapable of handling higher flow rates and pressures of fluid, which can further limit device performance and/or reduce sample throughput.

### SUMMARY

In accordance with the principles of the present disclosure, a microfluidic chip comprises a microchannel having a single inlet and a single outlet, a spiral section downstream from the single inlet, a straight section downstream from the spiral section, a detection section downstream from the straight section, and an expansion section downstream from the detection section and disposed between the detection section and the single outlet. The microchannel is configured to receive fluid having particles and/or cells. In addition, at least the straight section and the detection section are configured to orient particles within the detection section in an area away from sidewalls of the detection section and into one of a single particle stream or two particle streams. Further, the two particle streams immediately adjacent to each other appear as a single particle stream for optimized focusing and orientation of the particles in a focused stream within the microchannel.

In further accordance with the principles of the present disclosure, a microfluidic system comprises a microfluidic chip including a microchannel having a single inlet and a single outlet, a spiral section downstream from the single inlet, a straight section downstream from the spiral section, a detection section downstream from the straight section, and an expansion section downstream from the detection section and disposed between the detection section and the single outlet. The microchannel is configured to receive fluid having particles. The microfluidic system also comprises at least one detection means operatively coupled to the microfluidic chip, and the at least one detection means is configured to optically detect an orientation of at least one particle of the particles when disposed within the detection section of the microchannel for detection. So configured, at least the straight section and narrowing detection section of the microchannel and the pressure and flow rate of the fluid are configured to optimize focusing and orientation of the particles, orienting the particles away from sidewalls of the detection section and into one of a single particle stream or two particle streams. In addition, the two particle streams immediately adjacent to each other appear as a single particle stream, and at least one particle is parallel to a longitudinal axis of the detection section for optimized focusing and orientation of the particles within the microchannel.

In accordance with yet an another principle of the present disclosure, a method of focusing particles in a fluid within a microfluidic system comprises providing an inertial focusing microfluidic chip having a microchannel with a single inlet, a single outlet, a spiral section downstream from the single inlet, a straight section downstream from the spiral section, a narrowing detection section downstream from the straight section, and an expansion section downstream from the detection section and disposed between the detection section and the single outlet. In addition, the method also comprises flowing a fluid including particles into the single inlet, and orienting the particles away from sidewalls of the detection section and into one of single particle stream or two particle streams. The two particle streams immediately adjacent to each other appear as a single particle stream, and at least one particle is parallel to a longitudinal axis of the detection section for optimized focusing and detection of the particles. The method also comprises inertial focusing without additional introduction of a diluent fluid.

In accordance with yet another principle of the present disclosure, a microfluidic chip comprises a microchannel having a single inlet and a single outlet, a spiral section downstream from the single inlet, a detection section downstream from the spiral section, and a bridge disposed downstream from the detection section and coupling the detection section with the single outlet. The bridge is configured to collect a sample of cells at the single outlet and the detection section has a straight portion configured to orient particles within the detection section in an area away from sidewalls of the detection section and into one of a single particle stream or two particle streams. The two particle streams immediately adjacent to each other appear as a single stream for optimized focusing and orientation of particles within the microchannel.

In some examples, the particles may comprise sperm cells, and the sperm cells may comprise bovine or porcine sperm cells, all mammalian species sperm cells, and non-human animal sperm cells

In other examples, the optimized focusing and orientation of the particles may provide for detection by a detection means, and the detection may comprise a detection of a difference in DNA content in the particles, the difference in DNA content comprising one or more of: (1) approximately 4% difference in DNA content; or (2) the presence or absence of an X/Y chromosome. While this example is specific to X/Y chromosome detection, it is possible to label other DNA segments and use the same approach and still fall within the scope of the present disclosure.

In still other example, the detection means may comprise one from the group consisting of: (1) a photomultiplier tube; (2) an avalanche photodiode; and (3) a camera comprising a CCD.

In another example, the detection means may comprise an impedance detection means, the impedance detection means comprising a set/array of electrodes.

In yet another example, the detection is a detected difference in a fluorescence emission by the particles after interrogation by an interrogation means, and the interrogation means may comprise one or more of: (1) a source of electromagnetic radiation; or (2) a laser, the laser comprising one of a continuous wave laser or a pulsed laser.

In still another example, the detection section may comprise an interrogation region.

In another example, the detection section, may comprise an action region, and the action region may comprise a portion of the detection section for acting on a subset of particles based on the detection by a detection means.

In still another example, acting on the subset of particles may comprise irradiating each particle in the subset of particles by a source of electromagnetic radiation, and the source of electromagnetic radiation may include a laser having a pulsed laser, the irradiating may cause one of an ablation or a slicing and deactivating at least one particle of the particles within the fluid.

In still other examples, acting on the subset of particles may comprise diverting each particle in the subset of particles from the microchannel. Alternatively and/or additionally, acting on the subset of particles may comprise electroporating each particle in the subset of particles. Further, acting on the subset of particles may create an enriched population of particles, the enriched population of particles comprising a sexed semen sample.

In another example, the sexed semen sample may be configured to be inseminated in an animal and/or used to create an embryo, and the embryo may be configured to be implanted.

In still other examples, the detection section may have a width of any value in a range of 50 microns to 75 microns, such as a width of any one of about 50 microns, 55 microns, 60 microns, 65 microns, 70 microns, or 75 microns and a height of any value in a range of 25 microns to 75 microns, such as a width of any one of about 25 microns, 30 microns, 35 microns, 40 microns, 45 microns, 50 microns, 55 microns, 60 microns, 65 microns, 70 microns, or 75 microns, and the spiral section may have the same uniform height as the detection section.

In yet another example, the microchannel may comprise glass, and the microchannel may be configured to withstand fluid having a pressure of any value in a range of about 50 psi to about 100 psi, such as any one of about 50 psi, 55 psi, 60 psi, 65 psi, 70 psi, 75 psi, 80 psi, 85 psi, 90 psi, 95 psi or 100 psi and a flow rate of any value in a range of about 0.3 mL/min. to about 1.6 mL/min. In addition, an increase in a height of one or more of the microfluidic chip or the detection section may correspond to an increase in the flow rate, and the increased flow rate may correspond to an increase in optimized focusing of the particles.

In still other examples, the microfluidic chip may be an inside-out, spiral inertial focusing microfluidic chip, with a flow direction starting at the single inlet, through the spiral section, the straight section, the detection section, the expansion section and out to the single outlet.

In other examples, a reduced length of the straight section may reduce the resistance of the particles within the straight section, enabling an increased flow rate of fluid through the microchannel, and a reduced length of the detection section may enable a reduced operating pressure while achieving a consistent flow rate.

In still other examples, the parameters for optimized focusing may include one or more of: (1) an inner radius of the spiral section of the microchannel of about 1.0 mm and an outer radius of the spiral section of the microchannel of about 1.75 mm; and (2) a loop length of the spiral section of about 1.5 cm.

In addition, each of the spiral section and the straight section of the microchannel may include a width of about 75 microns and a height of about 45 microns, the detection section may include a width of about 50 microns and a height of about 45 microns, and the expansion section and single outlet may each include a width of about 500 microns and a height of about 300 microns.

In still other examples, one or more of: (1) cross-sectional dimensions of the spiral section and the straight section may be the same; (2) the cross-sectional dimensions of the detection section may be less than the cross-sectional dimensions of the spiral and straight sections; and (3) the expansion section and the outlet cross-sectional dimensions may be the same and greater than each of the spiral, straight, and detection sections.

In another example, the microchannel may further include a first tapering region disposed between the straight section and the detection section, and a second tapering region disposed between the detection section and the expansion section.

In yet another example, the microchannel may be configured to receive a media formulation such as fluid in which the particles are suspended the media formulation including a diluent fluid having a viscosity of one or more of about 0.00125 Pa*s, any value in a range of about 5% to about 25% greater than the viscosity of water, or about the same viscosity of water.

In some examples, the microfluidic system may be a cytometer system and further comprise one or more of a detection laser, a kill laser, a detector configured to detect light emitted from the particles, a field programmable gate array (FPGA) providing hardware control, and a computer control system, each of which may be operably coupled to the microfluidic chip. In addition, the computer control system may include a memory for data storage, a processor executable by the memory, and a user interface.

In other examples, the detection laser and the kill laser may have optics. The detection laser may be configured to cause fluorescence of dye in the particles, and the kill laser may be configured to fire on the particles in response to a timing instruction from one or more of a hardware control or a software control of the microfluidic system.

In still other examples, providing an inertial focusing microfluidic chip having a microchannel with a single inlet, a single outlet, a spiral section downstream from the single inlet, a straight section downstream from the spiral section, a narrowing detection section downstream from the straight section, and an expansion section downstream from the detection section may comprise providing the detection section having a width of any value in a range of about 50 microns to about 75 microns, such as any one of about 50 microns, 55 microns, 60 microns, 65 microns, 70 microns, or 75 microns and a height of any value in a range of about 25 microns to about 75 microns, such as any one of about 25 microns, 30 microns, 35 microns, 40 microns, 45 microns, 50 microns, 55 microns, 60 microns, 65 microns, 70 microns, or 75 microns. The spiral section may have the same uniform height as the detection section.

In another example, providing an inertial focusing microfluidic chip having a microchannel with a single inlet, a single outlet, a spiral section downstream from the single inlet, a straight section downstream from the spiral section, a narrowing detection section downstream from the straight section, and an expansion section downstream from the detection section may comprise providing one or more of: (1) an inner radius of the spiral section of about 1.0 mm and an outer radius of the spiral section of about 1.75 mm; and (2) a loop length of the spiral section of about 1.5 cm.

In still another example, providing an inertial focusing microfluidic chip having a microchannel with a single inlet, a single outlet, a spiral section downstream from the single inlet, a straight section downstream from the spiral section, a detection section downstream from the straight section, and an expansion section downstream from the detection section may further comprise providing one or more of: (1) each of the spiral section and the straight section having a width of about 75 microns and a height of about 45 microns; (2) the detection section having a width of about 50 microns and a height of about 45 microns; and (3) the expansion section and the single outlet each having a width of about 500 microns and a height of about 300 microns.

In yet another example, providing an inertial focusing microfluidic chip having a microchannel with a single inlet, a single outlet, a spiral section downstream from the single inlet, a straight section downstream from the spiral section, a detection section downstream from the straight section, and an expansion section downstream from the detection section may comprise providing cross-sectional dimensions of the spiral section and the straight section that are the same, providing cross-sectional dimensions of the detection section that are less than cross-sectional dimensions of the spiral and straight sections, and providing cross-sectional dimensions of the expansion section and the outlet that are the same and greater than each of the spiral, straight, and detection sections.

In still other examples, flowing a fluid including particles into the single inlet may comprise flowing a fluid including particles into the single inlet, the fluid having a pressure of any value in a range of about 50 psi to about 100 psi, such as any one of about 50 psi, 55 psi, 60 psi, 65 psi, 70 psi, 75 psi, 80 psi, 85 psi, 90 psi, 95 psi or 100 psi and a flow rate of any value in a range of 0.3 mL/min. to 1.6 mL/min.

In another example, the method may further comprise disposing a first tapering region between the straight section and the detection section and a second tapering region between the detection section and the expansion section.

In yet another example, flowing a fluid including particles into the single inlet may comprise flowing a fluid of particles including sperm cells into the single inlet.

In another example, flowing a fluid including particles into the single inlet may comprise flowing a fluid of particles including sperm cells into the single inlet, the sperm cells including bovine or porcine sperm cells.

In yet another example, the method may further comprise detecting particles within the detection section by a detection means by detecting a difference in DNA content in the particles, and the difference in DNA content may comprise one or more of: (1) approximately 4% difference in DNA content; or (2) the presence or absence of an X/Y chromosome.

In still other examples, detecting particles within the detection section by a detection means by detecting a difference in DNA content in the particles may comprise detecting particles by the detection means including one from the group consisting of: (1) a photomultiplier tube; (2) an avalanche photodiode; and (3) a camera comprising a CCD.

In other examples, detecting particles within the detection section by a detection means by detecting a difference in DNA content in the particles may comprise detecting particles by the detection means including an impedance detection means, and the impedance detection means may comprise a set/array of electrodes.

In another example, detecting particles within the detection section by a detection means by detecting a difference in DNA content in the particles may comprise detecting a detected difference in a fluorescence emission by the particles after interrogation by an interrogation means.

In still another example, detecting particles within the detection section by a detection means by detecting a difference in DNA content in the particles may comprise detecting a detected difference in a fluorescence emission by the particles after interrogation by an interrogation means may include one or more of: (1) a source of electromagnetic radiation; or (2) a laser, the laser comprising one of a continuous wave laser or a pulsed laser.

In another example, providing an inertial focusing microfluidic chip having a microchannel with a single inlet, a single outlet, a spiral section downstream from the single inlet, a straight section downstream from the spiral section, a detection section downstream from the straight section, and an expansion section downstream from the detection section may comprise providing a detection section having an interrogation region.

### BRIEF DESCRIPTION OF THE DRAWINGS

These and other features and advantages of the various exemplary embodiments disclosed herein will be better understood with respect to the following description and drawings, in which:

FIG. 1 is a block diagram of a microfluidic system according to one aspect of the present disclosure;

FIG. 2 is block diagram of portions of the microfluidic system of FIG. 1;

FIG. 3 is a perspective view of an exemplary microfluidic chip of the microfluidic system of FIG. 1;

FIG. 4A is a perspective view of a microchannel of the microfluidic chip of FIG. 3;

FIG. 4B is a side view of a portion of a microchannel of another microfluidic chip;

FIG. 4C is a perspective view of the portion of the microchannel of FIG. 4B;

FIG. 5A is a cross-sectional view taken along the line 5A-5A of FIG. 4 of a portion of a detection section of the microchannel of FIG. 4;

FIG. 5B is a top view of the portion of the detection section of the microchannel of FIG. 4;

FIG. 5C is a side view of the portion of the detection section of the microchannel of FIG. 4;

FIG. 5D is a perspective view of a flat cell in a portion of the detection section of the microchannel of FIG. 4;

FIG. 5E is a perspective view of a misaligned cell in a portion of the detection section of the microchannel of FIG. 4;

FIG. 5F is a perspective view of an edge-on cell in a portion of the detection section of the microchannel of FIG. 4;

FIG. 5G is another view of the portion of the detection section of the microchannel of FIG. 4;

FIG. 5H is a perspective view of a portion of the microchannel of FIG. 4, depicting an interrogation region of the microchannel;

FIG. 6 is a perspective view of another exemplary microfluidic chip that may be used with the microfluidic system of FIG. 1;

FIG. 7 is a perspective view of a microchannel of the microfluidic chip of FIG. 6;

FIG. 8 is a perspective view of another exemplary microfluidic chip that may be used with the microfluidic system of FIG. 1;

FIG. 9 is a perspective view of a microchannel of the microfluidic chip of FIG. 8;

FIG. 10 is a graph depicting a change in the width of a core stream of fluid in the microchannel of one or more of the exemplary microfluidic chips when increasing the pressure of the fluid at the inlet;

FIG. 11 is a graph depicting a change in orientation of particles within the fluid disposed in the microchannel of one or more of the exemplary microfluidic chips upon increasing the pressure of the fluid;

FIG. 12 is a graph depicting a change in Y and Z confinement of particles within the fluid disposed in the microchannel of the microfluidic chip of FIG. 7 upon increasing the pressure of the fluid;

FIG. 13 is a graph depicting a change in a core stream width of particles within the fluid disposed in the microchannel of the microfluidic chip upon increasing Reynold's number of the fluid;

FIG. 14 is a graph depicting a change in an orientation of particles within the fluid disposed in the microchannel of the microfluidic chip upon increasing the Reynold's number of the fluid; and

FIG. 15 is a graph depicting a change in an x-confinement of particles within the fluid disposed in the microchannel of the microfluidic chip upon increasing the Reynold's number of the fluid.

### DETAILED DESCRIPTION

Although the foregoing text sets forth a detailed description of numerous different embodiments, it should be understood that the legal scope of the invention may be defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Generally, a microfluidic system having a microfluidic chip with a microchannel is disclosed. In one example, the microfluidic system includes at least one detection means, such as a detection site or any other detection means. The microfluidic chip may be used with the as least one detection means. The microchannel is configured to receive fluid having particles. The geometry of sections of the microchannel and an increased pressure and flow rate of the fluid through the microchannel optimize focusing and orientation of the particles within the microchannel, orienting the particles away from the sidewall of the microchannel and into one or two particle streams immediately adjacent to each other (and thus appearing to be a single particle stream).

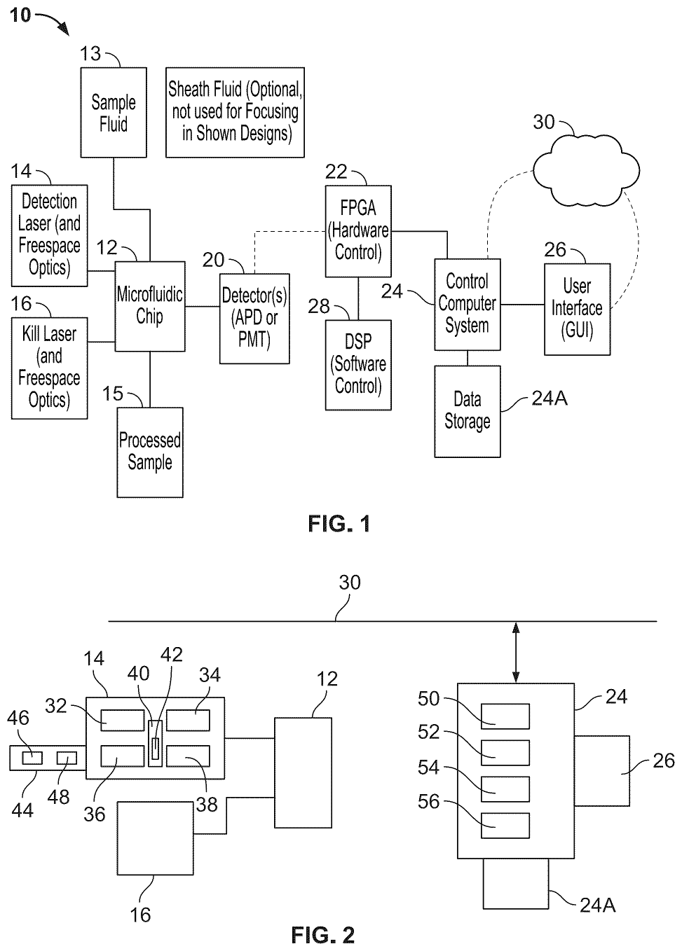

More specifically, and referring now to FIG. 1, a microfluidic system 10 according to several aspects of the present disclosure is depicted. The microfluidic system 10 includes a microfluidic chip 12 configured to receive fluid 13 having particles and at least one detection means 14 operatively coupled to the microfluidic chip 12. The at least one detection means 14 is configured to detect an orientation of at least one particle of the particles when disposed within a section of the microfluidic chip 12, such as a microchannel, as explained more below. In one example, the at least one detection means 14 is a detection laser 14 and may alternatively or additionally comprise different detection means, as explained more below. In this example, the detection laser 14 includes optics and is configured to cause fluorescence of dye in the particles of the fluid 13. It will be understood that the detection is about a characteristic of the particle and/or cell, such as a size, a shape, an amount of DNA, and/or a detectable morphology, for example. Orientation may also be detected or determined based upon the detected characteristic, as described above, but may not be the primary characteristic being detected in some example. Some detection means, such as multiple detectors, electrode-array impedance detection, CCD cameras/stroboscope, may provide better orientation detection than a combination EMR emitter and detector (e.g., APD, PMT), as will also be understood.

As also depicted in FIG. 1, the microfluidic system 10 further includes one or more of a kill laser 16, a detector 20 configured to detect light emitted from the particles, a field programmable gate array (FPGA) 22 providing hardware control, and a computer control system 24. Each of the kill laser 16, the detector 20, the FPGA 22, and the computer control system 24 is operatively coupled to the microfluidic chip 12. A graphical user interface (GUI) 26 may also be included in the microfluidic system 10, and the graphical user interface 26 may be coupled to or an integral part of the computer control system 24. The FPGA hardware control 22 may also include and/or be coupled to a DSP software control 28, and the computer control system 24 is operatively connected to one of a wireless network 30 or a wired network. So configured, data relative to the particles in the fluid 13 flowing through the microchannel of the microfluidic chip 14 may be stored within data storage 24A of the computer control system 24, for example, and/or transmitted via the wireless network 30 to one or more other computer systems, such as a remote computer system. In one example, the kill laser 16 includes optics and is configured to fire on the particles in the fluid 13 in response to a timing instruction from one or more of a hardware control, such as the FPGA 22 hardware control, or a software control, such as the DSP software control 28 of the microfluidic system 10. In yet another example, the microfluidic system 10 is a cytometer system. It will be appreciated that the microfluidic system 10 may alternatively and/or additionally include other types of systems different from the cytometer system and still fall within the scope of the present disclosure.

In operation, and as explained more below, the sample fluid 13 is flowed into the microchip 12 and focused by the microchannel (of the microchip 12) geometry and fluidic forces. The detection means 14 cause fluorescence of particles in the sample fluid 13, such as DNA intercalating dye in cells. In one example, the dye is Hoescht 33342 dye, and the detection means 14 is a 355 nm UV laser. The detection means 14 detects (e.g., stoke shifted) light emitted from the particles, such as cells. In one example, the FGPA hardware control 22 and DSP software control 28 determine a 4% difference and send action (timing) instruction to the kill laser 16 based on gating, for example. The kill laser 16 then fires on particles in the fluid 13, such as cells, based on the timing instruction. A histogram shown to a user on the GUI 26 reflects detected cells, a processing rate, a kill count, a dead cell percentage, and other information, as desired.

Referring now to FIG. 2, portions of the microfluidic system 10 of FIG. 1 are provided in block diagram form. As depicted, the at least one detection means 14 may include a detection laser 32. In addition, the at least one detection means may also include one from the group consisting of: (1) a photomultiplier tube 34; (2) an avalanche photodiode 36; or (3) a camera comprising a CCD 38. Each of the detection laser, the photomultiplier tube 34, the avalanche photodiode 36, and the camera 38 may be operatively coupled to the microfluidic chip 12, as depicted in FIG. 2. In another example, the at least one detection means comprises an impedance detection means 40, and the impedance detection means comprises a set and/or an array of electrodes 42. Further, the at least one detection means 14 may include a detected difference in a fluorescence emission by the particles in the fluid 13 after interrogation by an interrogation means 44. The interrogation means 44 may be integral with the at least one detection means 12, as depicted in FIG. 2, or separate from the at least one detection means 12. In another example, the interrogation means 44 may comprise one or more of a source 46 of electromagnetic radiation and/or a laser 48 comprising one of a continuous wave laser or a pulsed laser.

As further depicted in FIG. 2, the computer control system 24 may include one or more of a memory 50, such as a memory for data storage, a processor 52 executable by the memory 50, a user interface 54, which may include the graphical user interface 26, and a network interface 56 for connection with the wireless network 30 or a wired network, for example. The processor 52 may be a general processor, a digital signal processor, ASIC, field programmable gate array, graphics processing unit, analog circuit, digital circuit, or any other known or later developed processor. The processor 52 may operate pursuant to a profile stored in the memory 50 of the computer control system 24, for example. The memory 50 may be a volatile memory or a non-volatile memory. In addition, the memory 50 may include one or more of a read-only memory (“ROM”), random-access memory (“RAM”), a flash memory, an electronic erasable program read-only memory (“EEPROM”), or other type of memory. Further, the memory 50 may include an optical, magnetic (hard drive), or any other form of data storage. In addition, in other examples, the processor 52 may be a combination of a primary processor and a co-processor, e.g., x86 or x64 CPU for handling GUI, data storage, logging, and other operations, and a DSP for software-based hardware control.

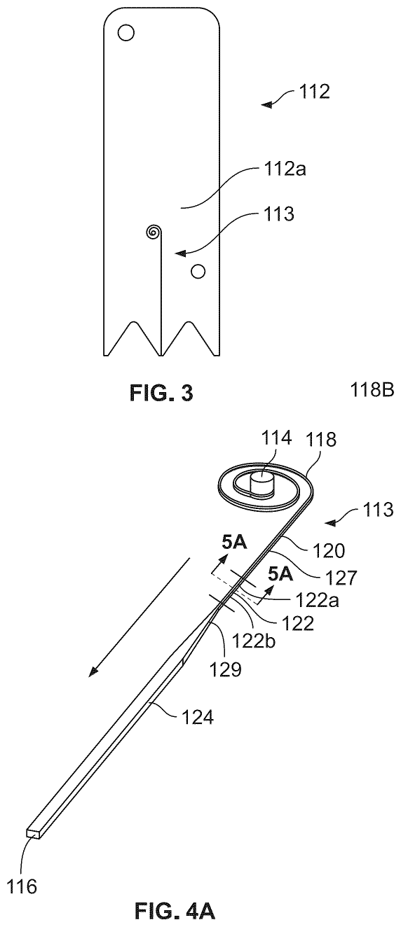

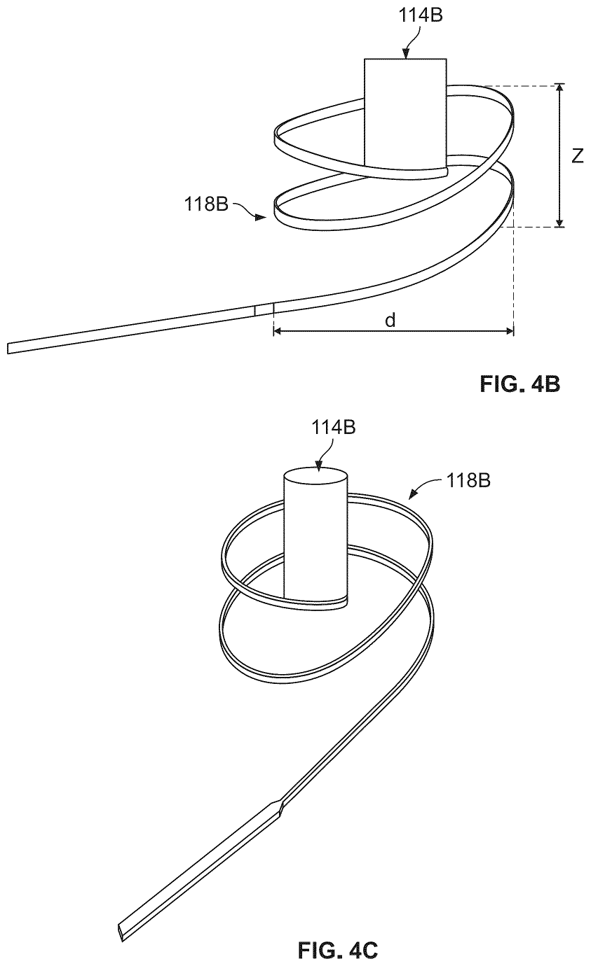

Referring now to FIG. 3, the microfluidic chip 12 of the microfluidic system 10 of FIGS. 1 and 2 may include various types of microfluidic chips. In one example, and as depicted in FIG. 4, the microfluidic chip 12 comprises a microfluidic chip 112. The microfluidic chip 112 includes a microchannel 113. The microfluidic chip 112 may include a body 112a comprising a substrate, and the microchannel 113 is disposed in the body 112a.

As depicted in FIG. 4A, a close-up view of the microchannel 113 of the microfluidic chip 112 of FIG. 3 is depicted. The microchannel 113 includes a single inlet 114, a single outlet 116, a spiral section 118 downstream from the single inlet 114, and a straight section 120 downstream from the spiral section 118. The microchannel 113 further includes a detection section 122, which is a narrowing detection section 122 in some examples, downstream from the straight section 120. An expansion section 124 is disposed downstream from the detection section 122 and between the detection section 122 and the single outlet 116.

Referring now to FIGS. 4B and 4C, a portion of a microchannel 113B of another embodiment of a microfluidic chip 112B is depicted. In this example, the microchannel 113B includes a spiral section 118B that is 3-dimensional in shape and having loops with the same diameter stacked one above the other. Any known additive manufacturing method and/or system may be used to 3D print the microchannel 113B of FIGS. 4B and 4C. This is in contrast to the spiral section 118 of the microchannel 113 of FIG. 4A, which is flat in shape, e.g., the loops are concentric. The microchannel 113B of FIGS. 4B and 4C may include one or more of the features described below relative to the microchannel 113 of FIG. 4A and still fall within the scope of the present disclosure.

As noted generally above, the microchannel 113 of the microfluidic chip 112 is configured to receive a media formulation, such as the fluid 13 having particles. In some examples, the particles comprise sperm cells, and the sperm cells may comprise bovine or porcine sperm cells. The particles are suspended in the fluid 13, and in one example, the media formulation, such as the fluid 13, includes a diluent fluid and comprises a viscosity of one or more of: (1) about 0.00125 Pa*s; (2) any value in a range of about 5% to about 25% greater than the viscosity of water; or (3) about the same viscosity of water. In some embodiments, the viscosity may be even higher, as long as the property of fluid is similar to water (Newtonian fluid). A higher viscosity would require higher pumping pressure to achieve the same results. In still other embodiments, the sample fluid 13 includes a first fluid comprising an analyte (e.g., ejaculate with sperm cells) and a second fluid comprising a diluent. The first/second fluids may be pre-mixed and loaded into a sample container, or may be stored separately and mixed immediately before entering the microfluidic chip. In other embodiments, the fluid 13 is a viscoelastic fluid and comprises a viscosity of one or more of: 1.8 mPats and 2.3 mPats for the concentration of 0.05% and 0.1% (wt/wt) of a commonly used poly(ethylene oxide) (PEO) (molecular weight 2 million g/mol) in a water solution. Generally, the viscosity of a viscoelastic fluid, for example, the PEO solution, would be dependent on the molecular weight of the PEO and the concentration of the PEO in the water solution, typically in the range of 1-6 mPa*s.

As further depicted in FIG. 4A, the detection section 122 includes an interrogation region 122a and an action region 122b for acting on a subset of particles based on the detection by the at least one detection means 14 described above. In one example, acting on the subset of particles comprises irradiating each particle in the subset of particles by a source of electromagnetic radiation 46, and the source of electromagnetic radiation 46 may include a laser, such as the laser 48 referred to above. The laser 48 may include a pulsed laser, and the irradiating may cause of one of an ablation or a slicing and deactivating of at least one particle of the particles within the fluid 13. In another example, acting on the subset of particles comprises diverting each particle in the subset of particles from the microchannel 113 or electroporating each particle in the subset of particles. In yet another example, acting on the subset of particles creates an enriched population of particles, and the enriched population of particles comprises a sample 15 (FIG. 1), such as a sexed semen sample. In this example, the sexed semen sample is configured to be inseminated in an animal and/or used to create an embryo, and the embryo is configured to be implanted.

In addition, in this example, the detection section 122 has a width of any value in a range of about 50 microns to about 75 microns, such as one of about 50 microns, 55 microns, 60 microns, 65 microns, 70 microns, or 75 microns and a height of any value in a range of about 25 microns to about 75 microns, such as any one of about 25 microns, 30 microns, 35 microns, 40 microns, 45 microns, 50 microns, 55 microns, 60 microns, 65 microns, 70 microns, or 75 microns. More generally, the microchannel 113 dimensions may be anywhere on this spectrum, such as the detection section 122 having a width of about 53 microns. In another example, the microchannel 113 may include smaller dimensions, such as the detection section 122 and/or other channels having a width and/or a height of any value in a range of about 15 microns to about 50 microns, such as using different fabrication techniques, and still fall within the scope of the present disclosure. Further, the spiral section 118 has the same uniform height as the detection section 122. In one example, and as provided in Table 1 below, the width of the detection section 112 is 50 microns and the height is 45 microns.

TABLE 1
Exemplary Parameters of Microchannel 113 of FIGS. 3 and 4

| Metrics | Microchannel 113 | |
|---|---|---|
| Cell Orientation (flat side up) | 98% | |
| Core stream width (Y_width) | 4.5 μm/20 μm | |
| 68%/100% | | |
| X spread | 4.24 μm/18.70 μm | |
| Pressure (psi) | 100 | psi |
| Sheath inlets | 0 (single inlet single outlet device) | |
| Average cell velocity | 7 | m/s |
| Flow rate | 0.586 | mL/min |

| | |
|---|---|
| Skew (mean) | 91% @ 10000 & 89.5% @ 15000 cells/s |
| Media | MD |
| Detection region dimensions | 50 um (width) × 45 um (height) |

In another example, the width of the detection section 122 is 50 microns and the height may be 35 microns, a uniform height of the spiral section 118 may be 35 microns, the flow rate may be 0.394 mL/min.

Further, the microchannel 113 comprises glass and is thus configured to withstand fluid 13 having a pressure of any value in a range of about 50 psi to about 100 psi, such as any one of about 50 psi, 55 psi, 60 psi, 65 psi, 70 psi, 75 psi, 80 psi, 85 psi, 90 psi, 95 psi or 100 psi and a flow rate of any value in a range of about 0.3 mL/min. to about 1.6 mL/min. In the example of Table 1 above, the pressure was 100 psi and the flow rate of the fluid 13 was 0.586 mL/min. Generally, an increase in a height of one or more of the microfluidic chip 112 or, more specifically, the detection section 122 corresponds to an increase in the flow rate of the fluid 13 flowing through the microchannel 113. In addition, the increased flow rate of fluid 13 corresponds to an increase in optimized focusing of the particles in the fluid 13.

A significant improvement of the spiral inertial microfluidic chip 112 as disclosed is that it produces only one streamline or two closely positioned streamlines (e.g., two streamlines that are close enough together to be within a single focal plane for a laser). Other types of spiral chips do not necessarily or inherently produce similar focusing effects. For example, many existing designs in the prior art were specifically configured to produce two or more streamlines that were not close together in order to facilitate particle separation. Additionally, unless properly configured, a spiral chip may typically produce two or more stream lines for the same particle type at different equilibrium positions within the channel. The specific configuration of the spiral channel 113 of the present disclosure provides for the one/two-in-proximity streamlines that are necessary for semen sexing (especially using EMR interrogation with Stoke-shift fluorescence detection and EMR deactivation/ablation). This property may also have uses in other applications outside of semen sexing.

In one example, the microfluidic chip 113 is an inside-out, spiral inertial focusing microfluidic chip. The flow direction starts at the single inlet 114, goes through the spiral section 118, the straight section 120, the detection section 122, the expansion section 124 and then out to the single outlet 116, as depicted in FIG. 4. Still further, a reduced length of the straight section 120 of the microchannel 113 reduces resistance of the particles within the straight section 120. This enables an increased flow rate of fluid 13 through the microchannel 113. In addition, a reduced length of the detection section 122 enables a reduced operating pressure while achieving a consistent flow rate of the fluid 13.

In another example, the microchannel 113 of the microfluidic chip 112 has the following parameters for optimized focusing. Specifically, an inner radius of the spiral section 118 of the microchannel 113 may be about 1.0 mm and an outer radius of the spiral section 118 of the microchannel 113 may be about 1.75 mm. Moreover, a loop length of the spiral section 118 may be about 1.5 cm.

In yet another example, and as depicted below in Table 2, the spiral section 118 and the straight section 120 of the microchannel 113 include a width of about 75 microns and a height of about 45 microns, the detection section 122 includes a width of about 50 microns and a height of about 45 microns, and the expansion section 124 and single outlet 116 each include a width of about 500 microns and a height of about 300 microns. In this example, the straight and detection portions 120, 122 have the same height, but different widths. The straight portion 120 is 6.92 mm in length and the detection section 122 (including the taper, such as to reduce the width from 75 microns to 50 microns) is 1.92 mm in length.

TABLE 2
Exemplary Parameters of Microchannel 113 of FIGS. 3 and 4

| Channel dimensions | Microchannel |
|---|---|
| Spiral portion | 75 um width × 45 um height |
| Straight portion | 75 um width × 45 um height × |
| | 5.29 mm length |
| Detection region | 50 um width × 45 um height |
| Outlet/expansion region | 500 um width × 300 um |
| | height |

In another example, the straight and detection potions may be combined into a single section. This combined section has a length of 5.29 mm, a width of 75 um, and a height of 45 um. The interrogation/detection and action regions within the channel would be within (near the end) of the single straight section. The shorter length and lack of width reduction reduces operating pressure for the microfluidic chip 112, for example.

More generally, the microchannel 113 of the microfluidic chip 112 may include one or more of: (1) cross-sectional dimensions of the spiral section 118 and the straight section 120 that are the same; (2) the cross-sectional dimensions of the detection section 122 that are less than the cross-sectional dimensions of the spiral and straight sections 118, 120; and (3) the expansion section 124 and the outlet 118 cross-sectional dimensions that are the same and greater than each of the spiral, straight, and detection sections 118, 120, 122.

Still further, the microchannel 113 further includes a first tapering region 127 disposed between the straight section 120 and the detection section 122, and a second tapering region 129 disposed between the detection section 122 and the expansion section 124, as also depicted in FIG. 4. In one example, the width of the first tapering region 127 decreases from a first width adjacent to the straight section 120 of about 75 microns to a second width more adjacent to the detection section 122 of about 50 microns, as depicted in Table 3 below. In addition, the second tapering region 129 increases from a first width adjacent to the detection section 122 of about 50 microns to a second width more adjacent to the outlet 116 of about 500 microns, as also provided in Table 3 below. In another example, the tapering region, such as the first tapering region or the second tapering region, may be in a Z direction as well.

TABLE 3
Exemplary Parameters of Tapering Regions
of Microchannel 113 of FIG. 4

| Tapering regions | Microchannel (113) | |
|---|---|---|
| 75 μm to 50 μm | −1.335° | (0.0233 mm) |
| 50 μm to 500 μm | 3.228° | (0.0564 mm) |

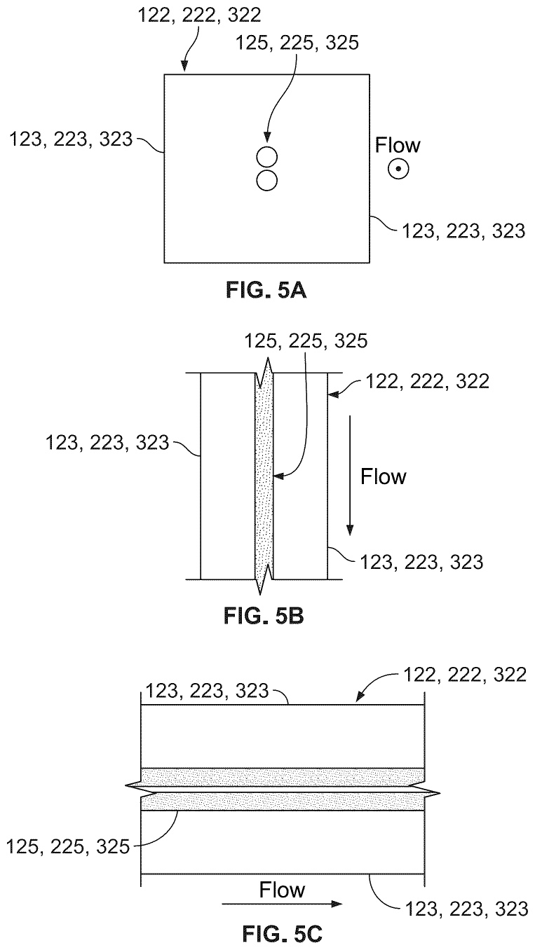

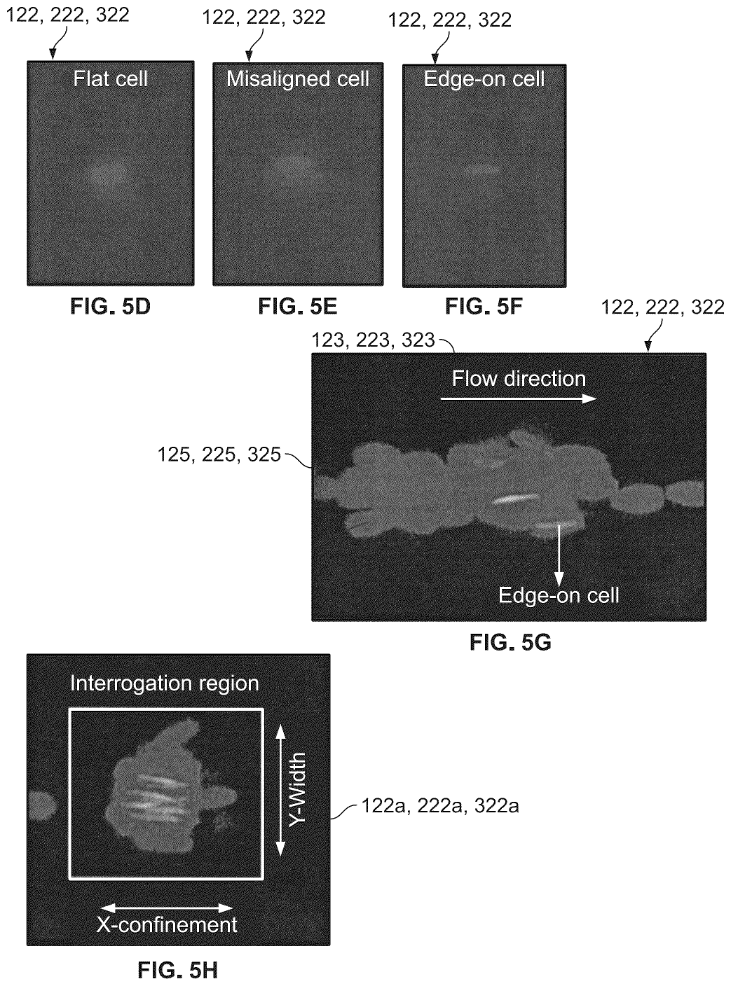

So configured, and as depicted in FIGS. 5A-5C, at least the straight section 120 and the detection section 122 are configured to orient particles within the detection section 122 in an area away from sidewalls 123 of the detection section 122 and into two particle streams 125. The two particle streams 125 may be immediately adjacent to each other, as depicted in FIG. 5A, or they may and can be separate by fluid, e.g., spaced in the Z direction, depending upon flow and sample parameters, for example. However, the two particle streams 125 appear as a single particle stream, as depicted in FIGS. 5B and 5C, for optimized focusing and orientation of the particles in a focused stream within the microchannel 113. In addition, the optimized focusing and orientation of the particles provides for detection by the at least one detection means 14 (FIGS. 1 and 2), and the detection comprises a detection of a difference in DNA content in the particles. The difference in DNA content may comprise one or more of: (1) approximately a 4% difference in DNA content; or (2) the presence or the absence of an X/Y chromosome, for example. For example, in some examples, the at least one detection means 14 may include one or more of the following embodiments: (1) detecting cells as being live or dead, and acting on a subset of the live cells based upon a further classification; (2) detecting the presence of an X or Y chromosome in cells, identifying cells comprising a Y chromosome, and acting on the Y chromosome bearing cells; (3) detecting the presence of an X or Y chromosome in cells, identifying cells not comprising an X chromosome, and acting on the not-X cells; (4) detecting the presence of an X or Y chromosome in cells, identifying cells comprising a X chromosome, and acting on the X chromosome bearing cells; and/or (5) detecting the presence of an X or Y chromosome in cells, identifying cells not comprising a Y chromosome, and acting on the not-Y cells. Further, although the above-described example is specific to X/Y chromosome detection, it will be understood that other DNA segments may be used in the systems and methods of the present invention and still fall within the scope of the present disclosure. For example, other DNA segments could be labeled and the same methods used to de-activate sperm cells with undesired traits.

Referring now to FIGS. 5D-5H, the orientation of particles, including cells, within the detection section 122 of the microfluidic channel 113 is depicted. The alignment and orientation of a flat cell of the particles 125 is depicted in FIG. 5D, a misaligned cell is depicted in FIG. 5E, and an edge-on cell is depicted in FIG. 5F. The alignments of these cells within the detection section 122 is obtained using a stroboscope, for example, or any other instrument capable of obtaining the same and/or similar images capturing the orientation and/or alignment of such particles and cells streaming through the detection section 122 of the microfluidic channel 113. Further, FIG. 5G depicts the particle stream 125 flowing through the detection section 122, with edge-on cells visible. Lastly, FIG. 5H depicts the particles 125, including cells, within the interrogation region 122a (also 222a and 322a as described in other examples below). In this example, the X-confinement and the Y-width of the particles 125 are depicted.

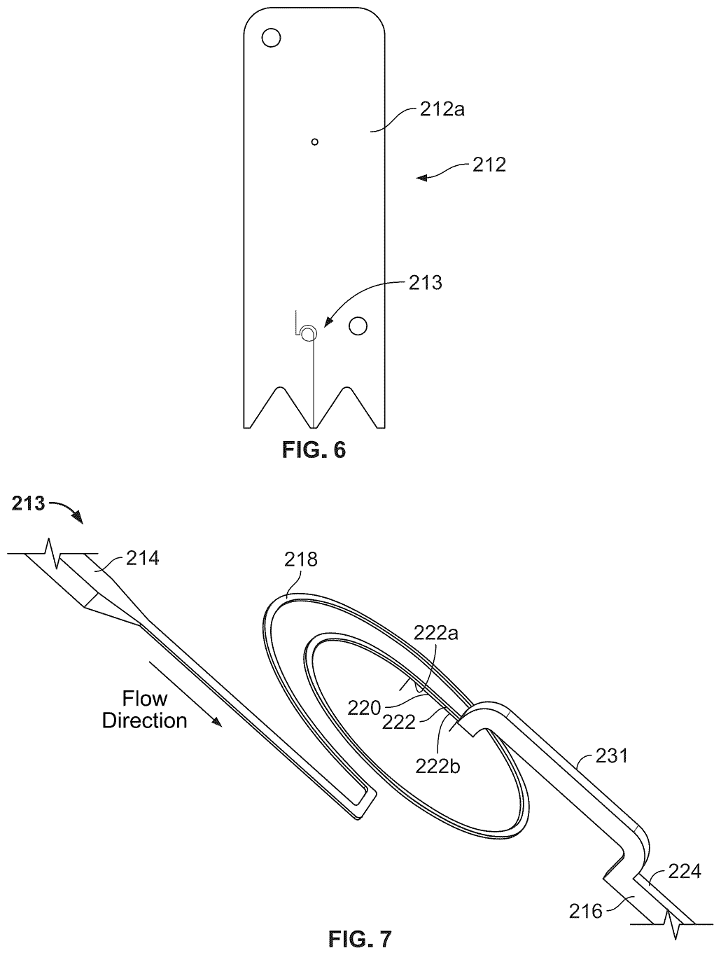

Referring now to FIGS. 6 and 7, in another example, the microfluidic chip 12 of the microfluidic system 10 (FIG. 1) may alternatively comprise a microfluidic chip 212, as depicted in FIG. 6. The microfluidic chip 212 includes a microchannel 213. Like the microfluidic chip 112, the microfluidic chip 212 may include a body 212a comprising a substrate, and the microchannel 213 is disposed in the body 212a. The microchannel 213 of FIGS. 6 and 7 is similar to the microchannel 113 of FIGS. 3 and 4 with some exceptions. For example, the microchannel 213 includes a bridge, as explained more below, which the microchannel 113 does not include. In addition, the microchannel 213 of the microfluidic device is an outside-in single inlet single outlet spiral microfluidic chip, which is different from the inside-out spiral microfluidic chip 112. As such, parts of the microchannel 213 similar to or the same as parts of the microchannel 113 have reference numbers 100 more than the reference numbers of the microchannel 113 and are not explained again here in depth for the sake of brevity.

Referring now to FIG. 7, a close-up view of the microchannel 213 of the microfluidic chip 212 of FIG. 6 is depicted. The microchannel 213 includes a single inlet 214, a single outlet 216, a spiral section 218 downstream from the single inlet 214, and a straight section 220 downstream from the spiral section 218. The microchannel 213 further includes a detection section 222, which is a narrowing detection section 222 in some examples. An expansion section 224 is disposed downstream from the detection section 122 and between the detection section 122 and the single outlet 116. In addition, a bridge 231 is disposed downstream from the detection section 222 between the detection section 222 and the expansion section 224. The bridge 231 is configured to collect a sample of cells from the fluid 13 at the single outlet 216. In addition, the detection section 222 includes the straight portion 220 configured to orient particles within the detection section 222 in an area away from sidewalls 223 of the detection section and into one of a single particle stream or two particle streams 225, as depicted in FIGS. 5A-5C. The two particle streams 225 are immediately adjacent to each other and appear as a single stream for optimized focusing and orientation of particles within the microchannel 213.

As noted, the microfluidic ship 212 is an outside-in spiral inertial focusing microfluidic chip. As such, the flow direction starts at the single inlet 214, goes through the spiral section 218, the detection section 220, the bridge 231, and then out to the single outlet 216.

The microchannel 213 of the microfluidic chip 212 is configured to receive a media formulation, such as the fluid 13 having particles. In some examples, the particles comprise sperm cells, and the sperm cells may comprise bovine or porcine sperm cells. The particles are suspended in the fluid 13, and in one example, the media formulation, such as the fluid 13, includes a diluent fluid having a viscosity of one or more of: (1) about 0.00125 Pa*s; (2) any value in a range of about 5% to about 25% greater than the viscosity of water; or (3) about the same viscosity of water.

As further depicted in FIG. 7, the detection section 222 may include an interrogation region 222a and an action region 222b for acting on a subset of particles based on the detection by the at least one detection means 14 described above. In one example, acting on the subset of particles comprises irradiating each particle in the subset of particles by a source of electromagnetic radiation 46, and the source of electromagnetic radiation 46 (FIG. 2) may include a laser, such as the laser 48 (FIG. 2) referred to above. The laser 48 may include a pulsed laser, and the irradiating may cause of one of an ablation or a slicing and deactivating of at least one particle of the particles within the fluid 13.

In this example, the spiral section 218 may have a uniform height of one of about 25 microns, 35 microns, or 45 microns, and the detection section 222 may have a width of any value in a range of about 50 microns to about 75 microns, such as any one of about 50 microns, 55 microns, 60 microns, 65 microns, 70 microns, or 75 microns and a height of any value in a range of about 25 microns to about 75 microns, such as any one of about 25 microns, 30 microns, 35 microns, 40 microns, 45 microns, 50 microns, 55 microns, 60 microns, 65 microns, 70 microns, or 75 microns. Generally, the height of the detection section 222 is approximately equal to the height of the spiral section 218.

In addition, and like the microchannel 113, the microchannel 213 comprises glass. The microchannel 213 is also configured to withstand fluid having a pressure of any value in a range of about 50 psi to about 100 psi, such as any one of about 50 psi, 55 psi, 60 psi, 65 psi, 70 psi, 75 psi, 80 psi, 85 psi, 90 psi, 95 psi or 100 psi and a flow rate of any value in a range of 0.3 mL/min. to 1.6 mL/min., improving focusing of the particles.

In one example, the microchannel 213 includes the detection section 222 having a width of 75 microns and a height of 25 microns, a pressure of 100 psi, a flow rate of the fluid, such as the fluid 13, of 390 μL/min (or 0.390 mL/min).

TABLE 4
Exemplary Parameters of Microchannel 213 of FIGS. 6 and 7

| Metrics | Microchannel (213) | |
|---|---|---|
| Cell Orientation (flat side up) | 97% | |
| Core stream width (Y_width) | 8 μm/26 μm | |
| 68%/100% | | |
| X spread | 8 μm/27 μm | |
| Pressure (psi) | 100 | psi |
| Sheath inlets | 0 (single inlet single outlet device) | |
| Average cell velocity | 3.5 | m/s |
| Flow rate | 0.39 | mL/min |

| | |
|---|---|
| Skew (mean) | 83% @ 10000 cell/s |
| Media | MD |
| Detection region dimensions | 75 um width × 25 um height |

In addition, in this example, a uniform height of the spiral section 218 of the microchannel 212 of 25 microns.

So configured, the microchannel 213 with the bridge 231 ensures a sample collection at the outlet 216. In addition, the parameters of the microchannel 213 result in optimization of the detection section 222 to ensure proper alignment of detection and the kill laser 16 (e.g., FIG. 1). Lastly, the flow conditions, such as the pressure and flow rate, of the fluid 13 flowing through the microchannel 213 are optimized for improved focusing performance.

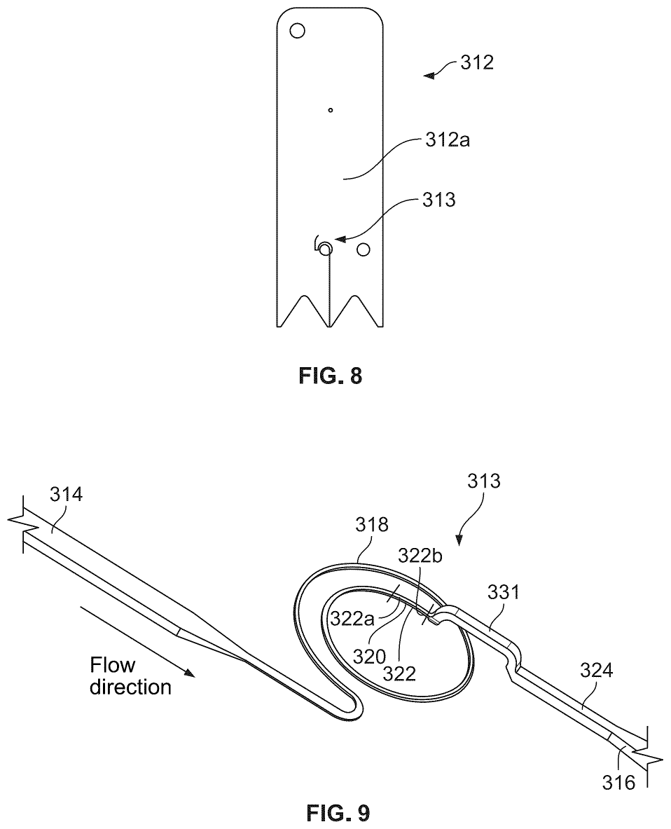

Referring now to FIGS. 8 and 9, in another example, the microfluidic chip 12 of the microfluidic system 10 (FIG. 1) may alternatively comprise a microfluidic chip 312, as depicted in FIG. 8. The microfluidic chip 312 includes a microchannel 313. Like the microfluidic chips 112, 212, the microfluidic chip 312 may include a body 312a comprising a substrate, and the microchannel 313 is disposed in the body 312a. The microchannel 313 of FIGS. 8 and 9 is similar to the microchannel 213 of FIGS. 6 and 7 with some exceptions. For example, the microchannel 313 includes a modified bridge to minimize vortices and clogging and all sharp corners of the microchannel 313 are smoothened and/or rounded (unlike the sharp corners of the microchannel 213). In addition, the depth of the microchannel 313 at the spiral section is larger than the depth of the spiral section 218 of the microchannel 213, as explained more below. As such, parts of the microchannel 313 similar to or the same as parts of the microchannel 213 have reference numbers 100 more than the reference numbers of the microchannel 213 and are not explained again in depth for the sake of brevity.

Referring now to FIG. 9, a close-up view of the microchannel 313 of the microfluidic chip 312 of FIG. 8 is depicted. The microchannel 313 includes a single inlet 314, a single outlet 316, a spiral section 318 downstream from the single inlet 314, and a detection section 322, which is a narrowing detection section 322 in some examples. The detection section 322 includes a straight section 320. An expansion section 324 is disposed downstream from the detection section 322 and between the detection section 322 and the single outlet 316. In addition, a bridge 331 is disposed downstream from the detection section 322 between the detection section 322 and the expansion section 324. The bridge 331 is configured to collect a sample of cells from the fluid 13 at the single outlet 316. In addition, the detection section 322 includes the straight portion 320 configured to orient particles within the detection section 322 in an area away from sidewalls 323 of the detection section 322 and into one of a single particle stream or two particle streams 325, as depicted in FIGS. 5A-5C. The two particle streams 325 are immediately adjacent to each other and appear as a single stream for optimized focusing and orientation of particles within the microchannel 313.

As noted, the microfluidic chip 312 is an outside-in spiral inertial focusing microfluidic chip, with a flow direction starting at the single inlet 314, through the spiral section 318, the detection section 322, the bridge 331, and out to the single outlet 316.

The microchannel 313 of the microfluidic chip 312 is configured to receive a media formulation, such as the fluid 13 having particles. In some examples, the particles again comprise sperm cells, and the sperm cells may comprise bovine or porcine sperm cells. The particles are suspended in the fluid 13, and in one example, the media formulation, such as the fluid 13, again may include a diluent fluid having a viscosity of one or more of: (1) about 0.00125 Pa*s; (2) any value in a range of about 5% to about 25% greater than the viscosity of water; or (3) about the same viscosity of water.

As further depicted in FIG. 9, the detection section 322 may include an interrogation region 322a and an action region 322b for acting on a subset of particles based on the detection by the at least one detection means 14 described above. In one example, acting on the subset of particles comprises irradiating each particle in the subset of particles by a source of electromagnetic radiation 46 (FIG. 2), and the source of electromagnetic radiation 46 may include a laser, such as the laser 48 (FIG. 2) referred to above. The laser 48 may include a pulsed laser, and the irradiating may cause of one of an ablation or a slicing and deactivating of at least one particle of the particles within the fluid 13.

In this example, the spiral section 318 may have a uniform height of any value in a range of about 25 microns to about 45 microns, such as any one of about 25 microns, 35 microns, or 45 microns, and the detection section 322 may have a width of any value in a range of about 50 microns to about 75 microns, such as any one of about 50 microns, 55 microns, 60 microns, 65 microns, 70 microns, or 75 microns and a height of any value in a range of about 25 microns to about 75 microns, such as any one of about 25 microns, 30 microns, 35 microns, 40 microns, 45 microns, 50 microns, 55 microns, 60 microns, 65 microns, 70 microns, or 75 microns. Generally, the height of the detection section 322 is approximately equal to the height of the spiral section 318.

In addition, and like the microchannels 113, 213, the microchannel 313 comprises glass. The microchannel 313 is also configured to withstand fluid having a pressure of any value in a range of about 50 psi to about 100 psi, such as any one of about 50 psi, 55 psi, 60 psi, 65 psi, 70 psi, 75 psi, 80 psi, 85 psi, 90 psi, 95 psi or 100 psi and a flow rate of any value in a range of 0.3 mL/min. to 1.6 mL/min., improving focusing of the particles.

In one example, the microchannel 313 includes the detection section 322 having a width of 75 microns and a height of 35 microns, a pressure of 100 psi, a flow rate of the fluid 13 of 567 μL/min (or 0.567 mL/min), as depicted in Table 5 below.

TABLE 5
Exemplary Parameters of Microchannel 313 of FIGS. 8 and 9

| Metrics | Microchannel (313) | |
|---|---|---|
| Cell Orientation (flat side up) | 97% | |
| Core stream width (Y_width) 68%/100% | 6.6 μm/23 μm | |
| X spread | 7.5 μm/24 μm | |
| Pressure (psi) | 100 | psi |
| Sheath inlets | 0 | |
| Average cell velocity | 6 | m/s |
| Flow rate | 0.567 | (mL/min) |

| | |
|---|---|
| Skew (mean) | Not measured |
| Media | MD |
| Detection region dimensions | 75 (width) × 35 um (height) |

In addition, in this example, a uniform height of the spiral section 318 of the microchannel 212 of 35 microns.

So configured, the microchannel 313 with the bridge 331 again ensures a sample collection at the outlet 316. In addition, the parameters of the microchannel 313 result in optimization of the detection section 322 to ensure proper alignment of detection and the kill laser 16 (e.g., FIG. 1). Lastly, the flow conditions, such as the pressure and the flow rate, of the fluid 13 flowing through the microchannel 313 are again optimized for improved focusing performance.

As indicated in each of Tables 1, 4, and 5, the media used within the microchannel 113, 213, 313 in these examples is MD as a diluent. However, upon further testing, results showed that MD as a diluent may have been responsible for issues in sample processing such as cell clumping and sample frothing. As a result, alternatives to MD as a diluent for the sample were also tested for all microchannels 113, 213, 313 of the microfluidic chips 12, 112, 212, 312 with a biocompatible diluent with a salt additive and comprising +2 μl/mL red food dye after experimental validation. The motility of particles in the fluid, such as cells, flowing through the microfluidic chips 112, 212, 312 with the new alternative diluent was not affected nor was particle, e.g., cell, viability.

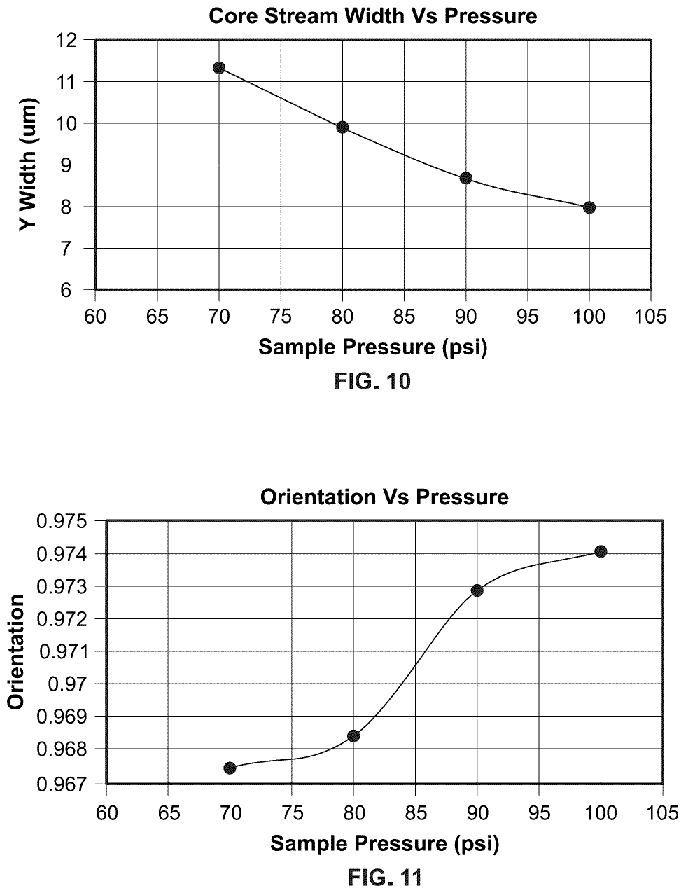

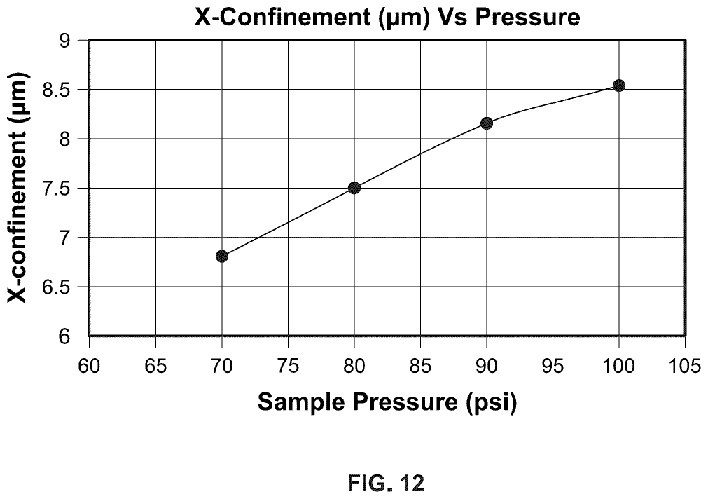

Referring now to FIGS. 10-12, using glass to fabricate each of the microchannels 113, 213, 313 described above and increasing the pressure of the fluid 13 flowing through the microchannels 113, 213, 313 improves focusing performance significantly. For example, and as depicted generally in FIG. 10, the core stream width decreases with increasing pressure, and the stream width in both the Y and Z directions is also dependent on ordination. In addition, the orientation of particles within the fluid desirably increases with increasing pressure, as depicted in FIG. 11, and X-confinement, such as X-spreading of the particles, also increases with increasing pressure, as depicted in FIG. 12.

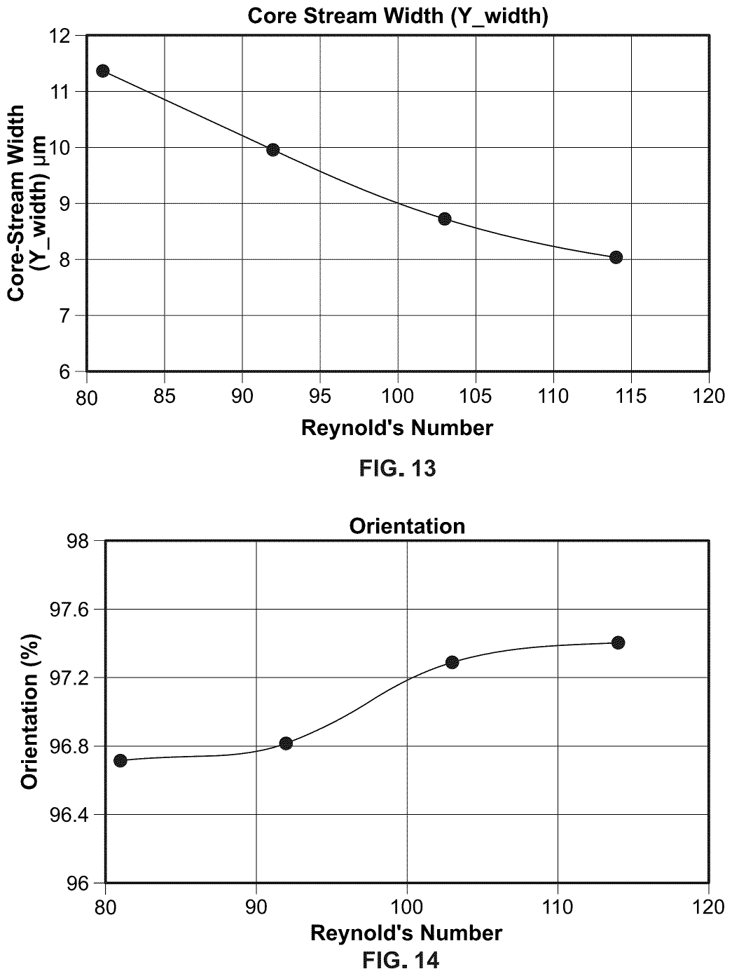

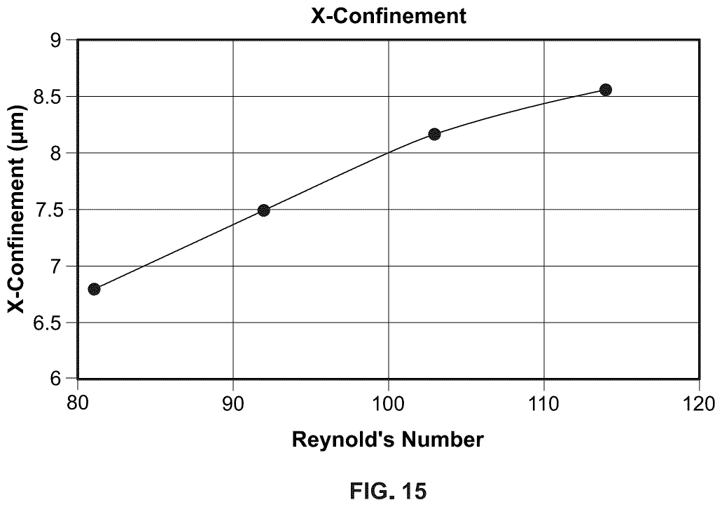

Referring now to FIGS. 13-15, the core-stream width, orientation, and X-confinement relative to an increasing Reynold's number are each depicted. More specifically, FIG. 13 shows that as the Reynold's number increases, the core-stream width decreases, and FIG. 14 shows that the orientation of particles within the fluid desirably increases with the increasing Reynold's number. Further, the x-confinement also desirably increases with increasing Reynold's number, as depicted in FIG. 15.

It will be appreciated that the microfluidic chips 112, 212, 312 capable of being used with the microfluidic system 10 of FIG. 1 may operate according to any one or more of the following methods described below. For example, and in one example, a method of focusing particles in a fluid within the microfluidic system 10 comprises providing an inertial focusing microfluidic chip 112, 212, 312 having a microchannel 113, 213, 313 with a single inlet 114, 214, 314, a single outlet 116, 216, 316, a spiral section 118, 218, 318 downstream from the single inlet 116, 216, 316, a straight section 120, 220, 320 downstream from the spiral section, a narrowing detection section 122, 222, 322, and an expansion section 124, 224, 324 downstream from the detection section 122, 222, 322 and disposed between the detection section 122, 222, 322 and the single outlet 116, 216, 316. The method further comprises flowing a fluid, such as the fluid 13, including particles into the single inlet 114, 214, 314, and then orienting the particles away from sidewalls 123, 223, 323 of the detection section 122, 222, 322 and into one of single particle stream or two particle streams 125, 225, 325, the two particle streams 125, 225, 325 immediately adjacent to each other appearing as a single particle stream, and at least one particle parallel to a longitudinal axis of the detection section 122, 222, 322 for optimized focusing and detection of the particles. The method still further includes inertial focusing without additional introduction of a diluent fluid.

In one example, providing an inertial focusing microfluidic chip 112, 212, 312 having a microchannel 113, 213, 313 with a single inlet 114, 214, 314, a single outlet 116, 216, 316, a spiral section 118, 218, 318 downstream from the single inlet 116, 216, 316, a straight section 120, 220, 320 downstream from the spiral section, a detection section 122, 222, 322, and an expansion section 124, 224, 324 downstream from the detection section 122, 222, 322 and disposed between the detection section 122, 222, 322 and the single outlet 116, 216, 316 comprises providing the detection section having a width of any one of about 50 microns, 55 microns, 60 microns, 65 microns, 70 microns, or 75 microns and a height of any one of about 25 microns, 30 microns, 35 microns, 40 microns, 45 microns, 50 microns, 55 microns, 60 microns, 65 microns, 70 microns, or 75 microns, the spiral section having the same uniform height as the detection section.

In another example, providing an inertial focusing microfluidic chip 112, 212, 312 having a microchannel 113, 213, 313 with a single inlet 114, 214, 314, a single outlet 116, 216, 316, a spiral section 118, 218, 318 downstream from the single inlet 116, 216, 316, a straight section 120, 220, 320 downstream from the spiral section, a detection section 122, 222, 322, and an expansion section 124, 224, 324 downstream from the detection section 122, 222, 322 and disposed between the detection section 122, 222, 322 and the single outlet 116, 216, 316 comprises providing one or more of: (1) an inner radius of the spiral section 118, 218, 318 of about 1.0 mm and an outer radius of the spiral section of about 1.75 mm; and (2) a loop length of the spiral section 118, 218, 318 of about 1.5 cm.

In yet another example, providing an inertial focusing microfluidic chip 112, 212, 312 having a microchannel 113, 213, 313 with a single inlet 114, 214, 314, a single outlet 116, 216, 316, a spiral section 118, 218, 318 downstream from the single inlet 116, 216, 316, a straight section 120, 220, 320 downstream from the spiral section, a detection section 122, 222, 322, and an expansion section 124, 224, 324 downstream from the detection section 122, 222, 322 and disposed between the detection section 122, 222, 322 and the single outlet 116, 216, 316 comprises providing one or more of: (1) each of the spiral section 118, 218, 318 and the straight section 120, 220, 320 having a width of about 75 microns and a height of about 45 microns; (2) the detection section 122, 222, 322 having a width of about 50 microns and a height of about 45 microns; and (3) the expansion section 124, 224, 324 and the single outlet 116, 216, 316 each having a width of about 500 microns and a height of about 300 microns.

In another example, providing an inertial focusing microfluidic chip 112, 212, 312 having a microchannel 113, 213, 313 with a single inlet 114, 214, 314, a single outlet 116, 216, 316, a spiral section 118, 218, 318 downstream from the single inlet 116, 216, 316, a straight section 120, 220, 320 downstream from the spiral section, a detection section 122, 222, 322, and an expansion section 124, 224, 324 downstream from the detection section 122, 222, 322 and disposed between the detection section 122, 222, 322 and the single outlet 116, 216, 316 comprises providing cross-sectional dimensions of the spiral section 118, 218, 318 and the straight section 120, 220, 320 that are the same, providing cross-sectional dimensions of the detection section 122, 222, 322 that are less than cross-sectional dimensions of the spiral and straight sections, and providing cross-sectional dimensions of the expansion section 124, 224, 324 and the outlet 116, 216, 316 that are the same and greater than each of the spiral, straight, and detection sections.

In addition, in another example, flowing a fluid including particles into the single inlet 116, 216, 316 comprises flowing a fluid including particles into the single inlet 116, 216, 316, the fluid having a pressure of any one of about 50 psi, 55 psi, 60 psi, 65 psi, 70 psi, 75 psi, 80 psi, 85 psi, 90 psi, 95 psi or 100 psi and a flow rate of any value in a range of 0.3 mL/min. to 1.6 mL/min.

In another example, the method may further comprise disposing a first tapering region 127 (FIG. 4) between the straight section 120 and the detection section 122, and a second tapering region 129 between the detection section 122 and the expansion section 124. Further, flowing a fluid, such as the fluid 13, including particles into the single inlet 114, 214, 314 may comprise flowing a fluid of particles including sperm cells into the single inlet 114, 214, 314, and the sperm cells may include bovine or porcine sperm cells.

In still another example, the method may further comprise detecting particles within the detection section 122, 222, 322 by at least one detection means 14, 32, 34, 36, 38, 40 by detecting a difference in DNA content in the particles. The difference in DNA content may comprise one or more of: (1) approximately 4% difference in DNA content; or (2) the presence or absence of an X/Y chromosome.

In another example, detecting particles within the detection section 122, 222, 322 by the at least one detection means 14, 32, 34, 36, 38, 40 by detecting a difference in DNA content in the particles may comprise detecting particles by the at least one detection means 14 including one from the group consisting of: (1) the photomultiplier tube 34 (FIG. 2); (2) the avalanche photodiode 36; and (3) the camera 38 comprising a CCD. Alternatively and/or additionally, detecting particles within the detection section 122, 222, 322 by the at least one detection means 14 by detecting a difference in DNA content in the particles may comprise detecting particles by the at least one detection means 14 including an impedance detection means 40 (FIG. 2), wherein the impedance detection means 40 comprising a set/array of electrodes 42. Alternatively and/or additionally, in another example, detecting particles within the detection section 122, 222, 322 by at least one detection means 14 by detecting a difference in DNA content in the particles may comprise detecting a detected difference in a fluorescence emission by the particles after interrogation by an interrogation means 44.

In yet another example, detecting particles within the detection section 122, 222, 322 by the at least one detection means 14 by detecting a difference in DNA content in the particles may comprise detecting a detected difference in a fluorescence emission by the particles after interrogation by the interrogation means 44, the interrogation means 44 including one or more of: (1) the source 46 of electromagnetic radiation; or (2) the laser 48, the laser 48 comprising one of a continuous wave laser or a pulsed laser, for example.

In another method, providing an inertial focusing microfluidic chip 112, 212, 312 having a microchannel 113, 213, 313 with a single inlet 114, 214, 314, a single outlet 116, 216, 316, a spiral section 118, 218, 318 downstream from the single inlet 116, 216, 316, a straight section 120, 220, 320 downstream from the spiral section, a narrowing detection section 122, 222, 322, and an expansion section 124, 224, 324 downstream from the detection section 122, 222, 322 and disposed between the detection section 122, 222, 322 and the single outlet 116, 216, 316 comprises providing a detection section 122, 222, 322 having an interrogation region 122a, 222a, 322a.

In yet another example, providing an inertial focusing microfluidic chip 112, 212, 312 having a microchannel 113, 213, 313 with a single inlet 114, 214, 314, a single outlet 116, 216, 316, a spiral section 118, 218, 318 downstream from the single inlet 116, 216, 316, a straight section 120, 220, 320 downstream from the spiral section, a narrowing detection section 122, 222, 322, and an expansion section 124, 224, 324 downstream from the detection section 122, 222, 322 and disposed between the detection section 122, 222, 322 and the single outlet 116, 216, 316 comprises providing the detection 122, 222, 322 section having an action region 122b, 222b, 322b, the action region 122b, 222b, 322b acting on a subset of particles based on the detection by the at least one detection means 14.

In another example, acting on the subset of particles comprises irradiating each particle in the subset of particles by the source 46 of electromagnetic radiation, the source 46 of electromagnetic radiation including a laser 48 having a pulsed laser, the irradiating causing one of an ablation or a slicing and deactivating at least one particle of the particles within the fluid, such as the fluid 13.

Further, acting on the subset of particles may comprise one or more of: (1) diverting each particle in the subset of particles from the microchannel 113, 213, 313; (2) electroporating each particle in the subset of particles; or (3) creating an enriched population of particles, the enriched population of particles comprising a sexed semen sample. The method may also comprise one or more of inseminating an animal using the sexed semen sample, creating an embryo using the sexed semen sample, and implanting the embryo created using the sexed semen sample.

At least in view of the foregoing, it will be understood that the aforementioned microfluidic system 10, microfluidic chips 12, 112, 212, 312, and related methods include several advantages. For example, increasing the flow rate of the fluid to a value within the desired range was critical to and significantly improved the performance of the microfluidic chip 112, 212, 312. In addition, increasing the channel height of the microchannel 113, 213, 313 to increase the flow rate of fluid, for example, increased the overall focusing performance of the microchannel 113, 213, 313. The bridge design minimizes vortex and clogging and ensures improved sample collection at the outlet 316 for the microchannel 313 of the microfluidic chip 312, for example. Moreover, optimization of the detection region 122, 222, 322 of each of the microchannel 113, 213, 313 ensured improved focusing and overall sexing performance of the microfluidic chip 112, 212, 312. Still further, optimizing the length of the microfluidic chip 112, 212, 312 ensured that the microfluidic chip 112, 212, 312 were compatible with other instruments during operation. Shortening of the straight section 120, 220, 320 and the detection region 122, 222, 322 reduces resistance, increasing the flow rate of fluid, such as the fluid 13, through the microchannel 113, 213, 313. This also may enable reduced operating pressure to achieve the same flow and velocity with little to no effect on skew, for example.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

This detailed description is to be construed as examples and does not describe every possible embodiment, as describing every possible embodiment would be impractical, if not impossible. One could implement numerous alternate embodiments, using either current technology or technology developed after the filing date of this application.

Variations of the specific configurations shown and described herein are within the scope of the principles of the present disclosure, and are included in all claims deriving therefrom.

1. A microfluidic chip comprising:
a microchannel having a single inlet and a single outlet, a spiral section downstream from the single inlet, a straight section downstream from the spiral section, a detection section downstream from the straight section, and an expansion section downstream from the detection section and disposed between the detection section and the single outlet, the microchannel configured to receive fluid having particles,
at least the straight section and the detection section are configured to orient particles within the detection section in an area away from sidewalls of the detection section and into one of a single particle stream or two particle streams, the two particle streams immediately adjacent to each other appearing as a single particle stream, for optimized focusing and orientation of the particles in a focused stream within the microchannel.

2. The microfluidic chip of claim 1, wherein the particles comprise sperm cells, and the sperm cells comprise bovine or porcine sperm cells.

3. (canceled)

4. The microfluidic chip of claim 1, wherein the optimized focusing and orientation of the particles provides for detection by a detection means, and wherein the detection comprises a detection of a difference in DNA content in the particles, the difference in DNA content comprising one or more of: (1) approximately 4% difference in DNA content; or (2) the presence or absence of an X/Y chromosome.

5. The microfluidic chip of claim 4, wherein one or more of: the detection means comprises at least one of: one from the group consisting of: (1) a photomultiplier tube; (2) an avalanche photodiode; and (3) a camera comprising a CCD; the detection means comprises an impedance detection means, the impedance detection means comprising a set/array of electrodes; or the detection is a detected difference in a fluorescence emission by the particles after interrogation by an interrogation means, the interrogation means comprises one or more of: (1) a source of electromagnetic radiation; or (2) a laser, the laser comprising one of a continuous wave laser or a pulsed laser.

6. (canceled)

7. (canceled)

8. (canceled)

9. The microfluidic chip of claim 1, wherein the detection section comprises one or more of: (1) an interrogation region; or (2) an action region, the action region comprising a portion of the detection section for acting on a subset of particles based on the detection by a detection means.

10. (canceled)

11. The microfluidic chip of claim 1, wherein acting on the subset of particles comprises one or more of: (1) irradiating each particle in the subset of particles by a source of electromagnetic radiation, the source of electromagnetic radiation including a laser having a pulsed laser, the irradiating causing one of an ablation or a slicing and deactivating at least one particle of the particles within the fluid; (2) diverting each particle in the subset of particles from the microchannel; (3) electroporating each particle in the subset of particles; and wherein acting on the subset of particles creates an enriched population of particles, the enriched population of particles comprising a sexed semen sample.

12. (canceled)

13. (canceled)

14. (canceled)

15. (canceled)

16. The microfluidic chip of claim 1, wherein the detection section has a width of any value in a range of about 50 microns to about 75 microns, such as any one of about 50 microns, 55 microns, 60 microns, 65 microns, 70 microns, or 75 microns, and a height of any value in a range of about 25 microns to about 75 microns, such as any one of about 25 microns, 30 microns, 35 microns, 40 microns, 45 microns, 50 microns, 55 microns, 60 microns, 65 microns, 70 microns, or 75 microns, the spiral section having the same uniform height as the detection section.

17. The microfluidic chip of claim 1, the microchannel comprising glass, the microchannel configured to withstand fluid having a pressure of any value in a range of about 50 psi to about 100 psi, such as any one of about 50 psi, 55 psi, 60 psi, 65 psi, 70 psi, 75 psi, 80 psi, 85 psi, 90 psi, 95 psi or 100 psi, and a flow rate of any value in a range of about 0.3 mL/min. to about 1.6 mL/min., wherein an increase in a height of one or more of the microfluidic chip or the detection section corresponds to an increase in the flow rate, and the increased flow rate corresponds to an increase in optimized focusing of the particles.

18. The microfluidic chip of claim 16, wherein the microfluidic chip is an inside-out, spiral inertial focusing microfluidic chip, with a flow direction starting at the single inlet, through the spiral section, the straight section, the detection section, the expansion section and out to the single outlet.

19. (canceled)

20. The microfluidic chip of claim 1, wherein parameters for optimized focusing include one or more of: (1) an inner radius of the spiral section of the microchannel of about 1.0 mm and an outer radius of the spiral section of the microchannel of about 1.75 mm; and (2) a loop length of the spiral section of about 1.5 cm.

21. The microfluidic chip of claim 1, wherein each of the spiral section and the straight section of the microchannel include a width of about 75 microns and a height of about 45 microns, the detection section includes a width of about 50 microns and a height of about 45 microns, and the expansion section and single outlet each include a width of about 500 microns and a height of about 300 microns.

22. The microfluidic chip of claim 1, wherein one or more of: (1) cross-sectional dimensions of the spiral section and the straight section are the same; (2) the cross-sectional dimensions of the detection section are less than the cross-sectional dimensions of the spiral and straight sections; and (3) the expansion section and the outlet cross-sectional dimensions are the same and greater than each of the spiral, straight, and detection sections.

23. The microfluidic chip of claim 1, the microchannel one or more of: (1) further including a first tapering region disposed between the straight section and the detection section, and a second tapering region disposed between the detection section and the expansion section; or (2) configured to receive a media formulation such as fluid in which the particles are suspended the media formulation including a diluent fluid having a viscosity of one or more of about 0.00125 Pa*s, any value in a range of about 5% to about 25% greater than the viscosity of water, or about the same viscosity of water.

24. (canceled)

25. A microfluidic system comprising:
a microfluidic chip including a microchannel having a single inlet and a single outlet, a spiral section downstream from the single inlet, a straight section downstream from the spiral section, a detection section downstream from the straight section, and an expansion section downstream from the detection section and disposed between the detection section and the single outlet, the microchannel configured to receive fluid having particles; and
at least one detection means operatively coupled to the microfluidic chip, the at least one detection means configured to optically detect an orientation of at least one particle of the particles when disposed within the detection section of the microchannel for detection,
wherein at least the straight section and narrowing detection section of the microchannel and the pressure and flow rate of the fluid are configure to optimize focusing and orientation of the particles, orienting the particles away from sidewalls of the detection section and into one of a single particle stream or two particle streams, the two particle streams immediately adjacent to each other appearing as a single particle stream, and at least one particle parallel to a longitudinal axis of the detection section for optimized focusing and orientation of the particles within the microchannel.

26. The microfluidic system of claim 25, wherein the microfluidic system is a cytometer system and further comprises one or more of a detection laser, a kill laser, a detector configured to detect light emitted from the particles, a field programmable gate array (FPGA) providing hardware control, and a computer control system, each of which is operably coupled to the microfluidic chip, the computer control system including a memory for data storage, a processor executable by the memory, and a user interface.

27. (canceled)

28. (canceled)

29. The microfluidic system of claim 25, the microchannel comprising glass and the microfluidic chip comprising a spiral inertial focusing microfluidic chip, with a flow direction starting at the single inlet, through the spiral section, the detection section, and out to the single outlet.

30. (canceled)

31. (canceled)

32. The microfluidic system of claim 25, wherein one or more of cross-sectional dimensions of the spiral section and the straight section are the same, cross-sectional dimensions of the detection section are less than cross-sectional dimensions of the spiral and straight sections, and the expansion section and the outlet cross-sectional dimensions are the same and greater than each of the spiral, straight, and detection sections.

33. The microfluidic system of claim 25, the microchannel further including a first tapering region disposed between the straight section and the detection section, and a second tapering region disposed between the detection section and the expansion section.

34. The microfluidic system of claim 25, wherein at least one of: (1) the fluid having particles is configured to have a pressure of any value in a range of about 50 psi to about 100 psi, such as any one of about 50 psi, 55 psi, 60 psi, 65 psi, 70 psi, 75 psi, 80 psi, 85 psi, 90 psi, 95 psi or 100 psi, and a flow rate of any value in a range of 0.3 mL/min. to 1.6 mL/min., improving focusing of the particles; and (2) the fluid having particles comprises a diluent fluid having a viscosity of one or more of about 0.00125 Pa*s, any value in a range of about 5% to about 25% greater than the viscosity of water, or about the same viscosity of water.

35. (canceled)

36. (canceled)

37. (canceled)

38. The microfluidic system of claim 25, wherein the optimized focusing of the particles provides for detection by the at least one detection means, and wherein the detection comprises a detection of a difference in DNA content in the particles, the difference in DNA content comprising one or more of: (1) approximately 4% difference in DNA content; or (2) the presence or absence of an X/Y chromosome.

39. The microfluidic system of claim 38, wherein the at least one detection means comprises at least one of: one from the group consisting of: (1) a photomultiplier tube; (2) an avalanche photodiode; and (3) a camera comprising a CCD; and an impedance detection means, the impedance detection means comprising a set/array of electrodes; and the at least one detection is a detected difference in a fluorescence emission by the particles after interrogation by an interrogation means.

40. (canceled)

41. (canceled)

42. (canceled)

43. The microfluidic system of claim 25, wherein the detection section comprises at least one of: (1) an interrogation region; and (2) an action region, the action region comprising a portion of the detection section for acting on a subset of particles based on the detection by the at least one detection means, and wherein acting on the subset of particles comprises at least one of: (1) irradiating each particle in the subset of particles by a source of electromagnetic radiation, the source of electromagnetic radiation including a laser having a pulsed laser, the irradiating causing one of an ablation or a slicing, and deactivating at least one particle of the particles within the fluid; (2) diverting each particle in the subset of particles from the microchannel; and (3) electroporating each particle in the subset of particles.

44.-70. (canceled)

71. A microfluidic chip comprising:
a microchannel having a single inlet and a single outlet, a spiral section downstream from the single inlet, a detection section downstream from the spiral section, and a bridge disposed downstream from the detection section and coupling the detection section with the single outlet, the bridge configured to collect a sample of cells at the single outlet and the detection section having a straight portion configured to orient particles within the detection section in an area away from sidewalls of the detection section and into one of a single particle stream or two particle streams, the two particle streams immediately adjacent to each other appearing as a single stream for optimized focusing and orientation of particles within the microchannel.

72. The microfluidic chip of claim 71, the spiral section having a uniform height of one of about 25 microns, 35 microns, or 45 microns, and the detection section having a width of any value in a range of about 50 microns to about 75 microns, such as any one of about 50 microns, 55 microns, 60 microns, 65 microns, 70 microns, or 75 microns, and a height of any value in a range of about 25 microns to about 75 microns, such as any one of about 25 microns, 30 microns, 35 microns, 40 microns, 45 microns, 50 microns, 55 microns, 60 microns, 65 microns, 70 microns, or 75 microns, the height of the detection section approximately equal to the height of the spiral section.

73. The microfluidic chip of claim 71, the microchannel one or more of: (1) comprising glass, the microchannel configured to withstand fluid having a pressure of any value in a range of about 50 psi to about 100 psi, such as one of about 50 psi, 55 psi, 60 psi, 65 psi, 70 psi, 75 psi, 80 psi, 85 psi, 90 psi, 95 psi or 100 psi, and a flow rate of any value in a range of 0.3 mL/min. to 1.6 mL/min., improving focusing of the particles; or (2) configured to receive a media formulation in which the particles are disposed, the media formulation including a diluent fluid having a viscosity of one or more of about 0.00125 Pa*s, any value in a range of about 5% to about 25% greater than the viscosity of water, or about the same viscosity of water.

74. (canceled)

75. (canceled)

76. (canceled)
